(19) United States
(12) Patent Application Publication
(10) Pub. No.: US 2015/0025112 A1
(43) Pub. Date: Jan. 22, 2015

(54) ORALLY DISINTEGRATING TABLET FORMULATIONS OF DONEPEZIL

(71) Applicant: SANOVEL ILAC SANAYI VE TICARET ANONIM SIRKETI, Istanbul (TR)

(72) Inventors: Ali Turkyilmaz, Istanbul (TR); Fatih Cengiz Aygul, Istanbul (TR); Devrim Celik, Istanbul (TR)

(21) Appl. No.: 14/334,930

(22) Filed: Jul. 18, 2014

(30) Foreign Application Priority Data
Jul. 19, 2013 (TR) TR2013-08801

(51) Int. Cl. A61K 31/445; A61K 47/10; A61K 47/02; A61K 9/20

(52) U.S. Cl. CPC A61K 31/445; A61K 9/2095; A61K 47/10; A61K 47/02
USPC 514/319

(57) ABSTRACT

The present invention relates to orally disintegrating tablet formulations of donepezil hydrochloride comprising magnesium aluminium silicate, and one or more pharmaceutically acceptable excipient and process for preparing such a formulation.

### CROSS REFERENCE TO RELATED APPLICATION

This application claims the priority of Turkish patent application, No. TR2013/08801, filed Jul. 19, 2013, the disclosure of which is incorporated by reference herein.

### TECHNICAL FIELD OF THE INVENTION

The present invention relates to orally disintegrating tablet formulations of donepezil hydrochloride comprising magnesium aluminium silicate, and one or more pharmaceutically acceptable excipient and process for preparing such a formulation.

### BACKGROUND OF THE INVENTION

Donepezil hydrochloride is a reversible inhibitor of the enzyme acetylcholinesterase, which is known as (±)-2,3-dihydro-5,6-dimethoxy-2-[[1-(phenylmethyl)-4-piperidinyl]methyl]-1H-inden-1-one hydrochloride and its chemical structure is shown in the Formula I.

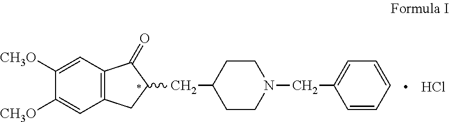

Donepezil hydrochloride is available for oral administration in conventional tablet formulations and orally disintegrating tablet formulations containing 5 or 10 mg of donepezil hydrochloride and indicated for the treatment of dementia and Alzheimer's Disease.

Various formulations and methods are already known for the preparation of orally disintegrating formulations of donepezil or pharmaceutically acceptable salts thereof. However, orally disintegrating formulations are becoming an increasingly important issue in the area of better patient compliance comparative to the conventional solid dosage forms for oral administration such as capsules and tablets, which are the most commonly used. In particular pediatric and geriatric patients, and patients with mental problems such as dementia and Alzheimer's disease, often experience difficulties in swallowing solid dosage forms. Besides, conventional solid dosage forms are not suitable for bedridden or busy and travelling patients, in case the patient may not have easy access to water. Thus, orally disintegrating compositions represent an alternative for such patients and provide for a better patient compliance with recommended pharmaceutical therapies.

Additionally oral administration of the drugs is difficult in patients having concomitant vomiting, nausea or diarrhoea. The orally disintegrating dosage form is one of the advantageous methods to deliver the drugs to such patients. By administering the orally disintegrating dosage forms, faster absorption of the drug occurs through buccal mucosa and it may reduce the first pass metabolism leading to better efficacy of the drug. This dosage form enhances the clinical effects of some drugs by leading to an increase in bioavailability and a reduction in side effects because of avoidance of first-pass liver metabolism.

It is known that, to develop orally disintegrating compositions are difficult because of several different reasons. A satisfied orally disintegrating dosage form needs to meet number of requirements. Firstly, it has to disintegrate in the oral cavity rapidly. Moreover, a premature release in the mouth could also lead to problems due to the often unpleasant taste of the active ingredient. Finally, these compositions should be very porous and should not be very hard. These porous compositions tend to be very sensitive to humidity so they may have some stability problems.

In order to meet all these above described requirements, careful selection of the excipients plays a significant role and they have to be chosen very carefully.

As a consequence, a need rises for orally disintegrating tablet formulations of donepezil hydrochloride and a process for preparing such formulations that overcome the problems as disintegration, taste and stability.

Present invention offers better stability, taste and disintegration rate of orally disintegrating tablet formulations of donepezil hydrochloride. According to present invention, orally disintegrating tablet formulations of donepezil hydrochloride does not comprise crospovidon in contrast to prior art and by using magnesium aluminum silicate, stability problems are solved and surprisingly better stability is gained. Also, specific combination of low viscosity hydroxypropyl cellulose (HPC-SSL) and high viscosity hydroxypropyl cellulose (L-HPC) yields a synergistic effect over the disintegration time and mechanical strength (such as; hardness and friability) of the orally disintegrating tablet formulation. Advantages and embodiments of the present invention will become apparent from the following description.

### DETAILED DESCRIPTION OF THE INVENTION

The main object of the present invention is to provide an improved orally disintegrating tablet formulation of donepezil hydrochloride comprising magnesium aluminum silicate which overcomes above described problems with using appropriate excipients.

According to this object the present invention is directed to an orally disintegrating tablet formulation of donepezil hydrochloride that is in an amount of 1.00 to 10.0% by weight, preferably it is 1.00 to 5.00% by weight of total tablet formulation, comprising magnesium aluminum silicate in an amount of between 1.00 to 15.00% by weight, preferably it is 2.00 to 10.00% by weight of total tablet formulation. Presence of magnesium aluminum silicate provides better stability for an orally disintegrating tablet formulation of donepezil hydrochloride which does not comprise crospovidon in contrast to prior art. It is found that crospovidon leads to stability problems due to impurities, thus cause to disintegrating problems during its shelf life period. It has surprisingly been found that magnesium aluminum silicate solves the stability problems and leads to desirable stability.

In another embodiment, the orally disintegrating tablet formulation of donepezil hydrochloride comprises hydroxypropyl cellulose with low and high viscosity.

In a further embodiment, the orally disintegrating tablet formulation of donepezil hydrochloride comprises hydroxypropyl cellulose having low viscosity which is between 2.0 and 2.9 mPa.s, (HPC-SSL), and hydroxypropyl cellulose having high viscosity which is between 6.0 and 10.0 mPa.s that is a grade of L-HPC that is LH-11.

The orally disintegrating tablet formulation of donepezil hydrochloride comprises low viscosity hydroxypropyl cellulose that is HPC-SSL in an amount of between 0.01 to 5.00% by weight, preferably it is 0.01 to 3.00% by weight of total tablet formulation. HPC-SSL having the lowest viscosity in all other grades of hydroxypropyl cellulose (HPC) is used as a binder.

Also, the orally disintegrating tablet formulation of donepezil hydrochloride comprises high viscosity hydroxypropyl cellulose that is L-HPC in an amount of between 1.00 to 20.00% by weight, preferably it is 1.00 to 10.00% by weight of total tablet formulation. L-HPC is used as a disintegrant.

In another embodiment, according to the orally disintegrating tablet formulation of donepezil hydrochloride, the ratio of HPC-SSL to L-HPC is between 1:10 and 10:1 by weight, preferably it is between 1:5 and 5:1 by weight, more preferably it is between 1:5 and 1:1 by weight.

It has surprisingly been found that the specific combination of HPC-SSL and L-HPC in the above-mentioned ratios creates a synergistic effect over the the disintegration time, mechanical strength (such as; hardness and friability) and compressibility of the orally disintegrating tablet formulation. L-HPC can absorb water fastly yielding powerful, high degree of swelling and so it leads to rapid disintegration of tablets and also using HPC-SSL with L-HPC provides optimum and desirable disintegration, better mechanical strength and better compressibility since presence of HPC-SSL preventing the immediate and undesirably fast disintegration of orally disintegrating tablet of donepezil hydrochloride. Therefore, due to the synergistic effect of L-HPC and HPC-SSL combination, the composition disintegrates in oral cavity in less than 60 seconds, preferably in less than 30 seconds.

In another embodiment, the orally disintegrating tablet formulation of donepezil hydrochloride comprises mannitol having the average particle size of 160 μm, Pearlitol 160C®, and mannitol having the average particle size of 360 μm, Pearlitol 400DC®. In this invention, the average particle size is measured by Malvern Particle Size analyzer based on Lazer Diffraction by using dry dispersion method. According to the calculation principle of the analyzer, the volume of the particles is converted into the volume of equivalent sphere and the result is the average diameters of these spheres which is volume moment mean D(4,3) value.

In the orally disintegrating tablet formulation of donepezil hydrochloride, the ratio of mannitol having the average particle size of 160 μm to mannitol having the average particle size of 360 μm is between 1:10 and 10:1 by weight, preferably it is between 1:5 and 5:1 by weight, more preferably it is between 1:5 and 1:1 by weight.

It is found that the specific combination of Pearlitol 160C® and Pearlitol 400DC® make a contribution to the obtaining better disintegration time and mechanical strength of the orally disintegrating tablet formulation. Therefore the hardness of the tablet is between 5 N to 50 N, preferably it is between 20 N to 30 N.

In another embodiment, the orally disintegrating tablet formulation of donepezil hydrochloride comprises magnesium aluminum silicate in an amount of between 1.00 to 15.00% by weight, HPC-SSL in an amount of between 0.01 to 5.00% by weight and L-HPC in an amount of between 1.00 to 20.00% by weight of total tablet formulation and one or more pharmaceutically acceptable excipient.

The orally disintegrating compositions of this invention also comprise sucralose as a sweetener to improve patient compliance. In prior art, it is know that aspartame is used mostly as sweetner but contradictory to the prior art we have found that the effect of sucralose as a sweetner in this formulation, not only helped to improve its taste but also increased the efficacy and the conveniency of the formulation because of its positive effects over the glycemic index. There are lots of disadvantages about aspartame and it has a limited usage if you have to use it every day and also there are several incompatibilities reported in literature and safety problems (Handbook of Pharmaceutical Excipients, Reymond C Rowe, Paul J Sheskey, Marian E Quinn, sixth edition, pages 48-50). Thus, sucralose has an important role in this aspect and even if it is used in low amounts it has a synergistic taste improvement with mannitol which is also very important issue in orally disintegrating tablet formulations. According to this object of the present invention sucralose is present in an amount of between 0.001 to 2.00% by weight, preferably it is 0.01 to 1.00% by weight.

In a further embodiment the orally disintegrating tablet formulation of donepezil hydrochloride further comprises one or more pharmaceutically acceptable excipients selected from the group comprising lubricants, glidants and disintegrants.

Suitable lubricants may comprise but not limited to sodium stearyl fumarate, magnesium stearate, polyethylene glycol, stearic acid, metal stearates, boric acid, sodium chloride benzoate and acetate and the like and mixtures thereof, preferably the lubricant is sodium stearyl fumarate. In one aspect, sodium stearyl fumarate is present in an amount of 0.1 to 5.00%, preferably it is 1.0 to 8.0% by weight of the total tablet formulation.

Suitable glidants may comprise but not limited to colloidal silicon dioxide, calcium silicate, magnesium silicate and talc and the like and mixtures thereof, preferably the glidant is colloidal silicon dioxide. In one aspect, colloidal silicon dioxide is present in an amount of 0.01 to 5.00%, preferably it is 0.1 to 5.0% by weight of the total tablet formulation.

Suitable disintegrants other than L-HPC may comprise but not limited to microcrystalline cellulose, croscarmellose sodium, starch and pregelatinized starch and the like and mixtures thereof, preferably the second disintegrant is sodium starch glycolate. In one aspect, sodium starch glycolate is present in an amount of 0.1 to 10.0% preferably it is 1.00 to 8.00% by weight of the total tablet formulation.

As it is mentioned above, to develop orally disintegrating compositions are difficult because of several different reasons. A satisfied orally disintegrating dosage form needs to fulfill the requirements of disintegration, taste and stability. To fulfill all these requirements the formulation for a specific drug needs to be adapted in particular by a careful selection of the excipients used.

In order to minimize the disintegration time and maximise the mechanical resistance of the tablets of this invention, this orally disintegrating tablet formulation has been designed, consisting the followings:

a. 1.00 to 10.0% by weight of donepezil hydrochloride,

b. 1.00 to 15.00% by weight of magnesium aluminum silicate,

c. 0.01 to 5.00% by weight low viscosity HPC (HPC-SSL),

d. 1.00 to 20.00% by weight high viscosity HPC (L-HPC),

e. 10.00 to 80.00% by weight mannitol having the average particle size of 160 μm, (Pearlitol 160C®),

f. 5.00 to 50.00% by weight mannitol having the average particle size of 360 μm, (Pearlitol 400DC®),

g. 0.001 to 2.00% by weight sucralose,

h. 0.1 to 5.00% by weight sodium stearyl fumarate,

i. 0.1 to 10.0% by weight sodium starch glycolate,

j. 0.01 to 5.00% by weight collodial silicon dioxide.

The process of the present invention for preparing the orally disintegrating tablet formulation of donepezil hydrochloride comprises the following steps;

a. mixing low viscosity HPC and distilled water until having homogenous mixture,

b. mixing donepezil hydrochloride, mannitol having the average particle size of 160 μm, magnesium aluminum silicate and sucralose,

c. adding the granulation solution in (a) to the powder mixture in (b) and granulation process is conducted,

d. the wet granules are sieved and dried and and dried granules are sieved again,

e. adding mannitol mannitol having the average particle size of 360 μm, high viscosity HPC, sodium starch glycolate, collodial silicon dioxide to the sieved granules and mixing,

f. adding the sieved sodium stearyl fumarate to the mixture in (e) and mixing until having homogenous mixture,

g. compressing the final mixture to form tablets.

In a further aspect, the present invention shows that it is possible to have a significant influence on the disintegration rate of the tablet by modifying the dimensions and shape of the tablet. In general, as the tablet becomes thinner and have higher porosity, the orally disintegrating composition will be weakened faster when it contacts with saliva, because the disintegration process is produced after wetting all the surface of the tablet via capillary action. Also, any shape which maximizes the contact surface with the saliva may produce a significant reduction in disintegration time. Therefore, the preferred shape of the orally disintegrating tablet is a round shape.

This invention is further defined by reference to the following examples. Although the examples are not intended to limit the scope of the present invention, it should be considered in the light of the description detailed above.

### EXAMPLE 1

### Orally Disintegrating Donepezil Hydrochloride Tablets

| Ingredients | Amount (mg) |
|---|---|
| Donepezil hydrochloride | 10.00 |
| Pearlitol 160C ® | 134.00 |
| Magnesium aluminum silicate | 12.60 |
| Sucralose | 1.5 |
| Hydroxypropyl cellulose (SSL) | 5.00 |
| Hydroxypropyl cellulose (LH-11) | 14.00 |
| Pearlitol 400DC ® | 80.50 |
| Sodium starch glycolate | 14.00 |
| Collodial silicon dioxide | 1.40 |
| Sodium stearyl fumarate | 7.00 |
| Distilled water | k.m. |
| Total tablet weight | 280.0 |

### EXAMPLE 2

### Orally Disintegrating Donepezil Hydrochloride Tablets

| Ingredients | Amount (mg) |
|---|---|
| Donepezil hydrochloride | 5.00 |
| Pearlitol 160C ® | 139.00 |
| Magnesium aluminum silicate | 12.60 |
| Sucralose | 1.50 |
| Hydroxypropyl cellulose (SSL) | 5.00 |
| Hydroxypropyl cellulose (LH-11) | 14.00 |
| Pearlitol 400DC ® | 80.50 |
| Sodium starch glycolate | 14.00 |
| Collodial silicon dioxide | 1.40 |
| Sodium stearyl fumarate | 7.00 |
| Distilled water | k.m. |
| Total tablet weight | 280.0 |

1. An orally disintegrating tablet formulation comprising donepezil hydrochloride, magnesium aluminum silicate and one or more pharmaceutically acceptable excipients, wherein said formulation is crospovidon free.

2. The orally disintegrating tablet formulation according to claim 1, wherein the magnesium aluminum silicate is present in an amount of 1.00% to 15.0% by weight of the formulation.

3. The orally disintegrating tablet formulation according to claim 1, further comprising low viscosity hydroxypropyl cellulose and high viscosity hydroxypropyl cellulose.

4. The orally disintegrating tablet formulation according to claim 3, wherein the viscosity of the low viscosity hydroxypropyl cellulose is between 2.0 mPa.s and 2.9 mPa.s and the viscosity of the high viscosity hydroxypropyl cellulose is between 6.0 mPa.s and 10.0 mPa.s.

5. The orally disintegrating tablet formulation according to claim 3, wherein the ratio of the low viscosity hydroxypropyl cellulose to the high viscosity hydroxypropyl cellulose is between 1:10 and 10:1 by weight.

6. The orally disintegrating tablet formulation according to claim 1, wherein
a. the magnesium aluminum silicate is in an amount of 1.00% to 15.00% by weight of the formulation,
b. the low viscosity hydroxypropyl cellulose is in an amount of 0.01% to 5.00% by weight of the formulation, and
c. the high viscosity hydroxypropyl cellulose is in an amount of 1.00% to 20.00% by weight of the formulation.

7. The orally disintegrating tablet formulation according to claim 1, further comprising two different mannitols, wherein one of the mannitols has an average particle size of 160 μm and the other mannitol has an average particle size of 360 μm.

8. The orally disintegrating tablet formulation according to claim 7, wherein the ratio of the mannitol having an average particle size of 160 μm to the mannitol having an average particle size of 360 μm is between 1:10 and 10:1 by weight.

9. The orally disintegrating tablet formulation according to claim 1, wherein the formulation disintegrates in an oral cavity in less than 60 seconds after oral administration.

10. The orally disintegrating tablet formulation according to claim 1, wherein the hardness of the tablet is between 5 N and 50 N.

11. The orally disintegrating tablet formulation according to claim 3 consisting of;
a. 1.0 to 10.0% by weight of the donepezil hydrochloride,
b. 1.00 to 15.00% by weight of the magnesium aluminum silicate,
c. 0.01 to 5.00% by weight of the low viscosity hydroxypropyl cellulose,
d. 1.00 to 20.00% by weight of the high viscosity hydroxypropyl cellulose,
e. 10.00 to 80.00% by weight of mannitol having an average particle size of 160 μm,
f. 5.00 to 50.00% by weight of mannitol having an average particle size of 360 μm,
g. 0.001 to 2.00% by weight of sucralose,
h. 0.1 to 5.00% by weight of sodium stearyl fumarate,
i. 0.1 to 10.0% by weight of sodium starch glycolate, and
j. 0.01 to 5.00% by weight of colloidal silicon dioxide.

12. (canceled)

13. The orally disintegrating tablet formulation of claim 9, wherein the formulation disintegrates in the oral cavity in less than 30 seconds after oral administration.

14. The orally disintegrating tablet formulation of claim 10, wherein the hardness of the tablet is between 20 N and 30 N.

15. The orally disintegrating tablet formulation according to claim 4, wherein the ratio of the low viscosity hydroxypropyl cellulose to the high viscosity hydroxypropyl cellulose is between 1:10 and 10:1 by weight.

16. The orally disintegrating tablet formulation according to claim 3, wherein the one or more pharmaceutically acceptable excipients further comprise mannitol having an average particle size of 160 μm, magnesium aluminum silicate, sucralose, mannitol having an average particle size of 360 μm, sodium starch glycolate, colloidal silicon dioxide and sodium stearyl fumarate.

17. A process for preparing orally disintegrating tablet formulation according to claim 16 comprising;
a. mixing low viscosity hydroxypropyl cellulose and distilled water until a homogenous mixture is obtained,
b. mixing donepezil hydrochloride, mannitol having an average particle size of 160 μm, magnesium aluminum silicate and sucralose,
c. adding the granulation solution obtained in step (a) to the powder mixture obtained from step (b) and then conducting granulation to obtain wet granules,
d. sieving and drying the wet granules to obtain dried granules and then sieving the dried granules,
e. adding mannitol having an average particle size of 360 μm, high viscosity hydroxypropyl cellulose, sodium starch glycolate, colloidal silicon dioxide to the sieved granules and mixing,
f. adding sieved sodium stearyl fumarate to the mixture obtained from step (e) and mixing until a homogenous mixture is obtained, and
g. compressing the product of step (f) to form tablets.
